(19) United States
(12) Patent Application Publication
(10) Pub. No.: US 2025/0228746 A1
(43) Pub. Date: Jul. 17, 2025

(54) NASOJEJUNAL FEEDING TUBE WITH EXPANDABLE GEOMETRY FOR RETENTION

(71) Applicant: BOSTON SCIENTIFIC SCIMED, INC., Maple Grove, MN (US)

(72) Inventors: John B. GOLDEN, Norton, MA (US); Jeff GRAY, Sudbury, MA (US); John Thomas FAVREAU, Charlton, MA (US); Peter L. DAYTON, Brookline, MA (US); Ryan V. WALES, Northborough, MA (US); Paul SMITH, Smithfield, RI (US); Kapil GUPTA, Boston, MA (US); Laura Elizabeth CHRISTAKIS, Framingham, MA (US); Rosangel Aimee RAMOS ESPINOZA, Boston, MA (US); Nathan Thomas CUMMINGS, Worcester, MA (US)

(21) Appl. No.: 19/016,046

(22) Filed: Jan. 10, 2025

Related U.S. Application Data
(60) Provisional application No. 63/621,823, filed on Jan. 17, 2024.

(51) Int. Cl. A61J 15/00

(52) U.S. Cl. CPC A61J 15/0019; A61J 15/0046; A61J 15/0069

(57) ABSTRACT

A device for administering a fluid to a patient anatomy includes a flexible catheter. The catheter includes a lumen extending therethrough. The catheter is sized and shaped to extend from a proximal end outside a human body to a distal end that can be guided to a target location. A distal portion of the catheter includes one or more exit ports communicating with a first lumen for administering the fluid. A deformable portion of the catheter is formed so that, in a first state, the deformable portion assumes a first shape suitable for guiding the catheter to the target location and, in a second state, the deformable portion assumes a second shape in which the deformable portion curves to extend in at least one radial direction for engaging an inner wall of the target location to anchor the catheter in the target location while administering the fluid.

### PRIORITY CLAIM

The present disclosure claims priority to U.S. Provisional Patent Application Ser. No. 63/621,823 filed Jan. 17, 2024; the disclosure of which is incorporated herewith by

### FIELD

The present disclosure relates to tubes and/or catheters with retention features for anchoring in the small bowel.

### BACKGROUND

Pyloric stenosis constitutes a narrowing of the gastric outlet (pylorus) causing difficulties in passing food from the stomach into the small bowel. This can lead to a gastric outlet obstruction (stricture). The inability to pass food into the small bowel can reduce or completely impede a person's ability to digest food and absorb nutrients. There are several solutions to this problem, including placing a nasojejunal (NJ) feeding tube in the small bowel, e.g., jejunum. Another solution includes the creation of a surgical gastro-jejunostomy bypass bridging the stomach and the jejunum to bypass the area of the stricture. NJ tubes can be used for assisting endoscopic ultrasound guided gastroenterostomy (EUS-GE) procedures by instilling contrast or another fluid in the jejunum. In one example, contrast is provided via the NJ tube to help make visible a target to an operating physician, e.g., for a procedure including piercing the stomach and the jejunum and placing a stent (e.g., AXIOS stent) therebetween to bypass the pylorus.

One issue with NJ tubes is that the tube may drift out of position during use so that fluids discharged by the tube are provided to an unintended location (i.e., as the outlet of the tube is no longer located in a target location). In one example, the NJ tube may migrate proximally during use. In feeding tube applications, this proximal drift may cause nutrients to be provided further upstream in the small bowel, or in the stomach proximal to the stricture, which would eliminates the benefits of the tube and may cause issues with digestion. In EUS-GE applications, the drift of the NJ tube can cause fluids (e.g., contrast fluid) to be provided to a location spaced from an intended target area. The stents for bypassing the pylorus may be mis-deployed if the physician is confused or misdirected due to the contrast fluid being provided to an unintended location during gastro-jejunostomy procedures.

### SUMMARY

The present disclosure relates to a device for administering a fluid to a patient anatomy. The device includes a flexible catheter including a lumen extending therethrough, the catheter being sized and shaped to extend from a proximal end outside a human body to a distal end that can be guided to a target location within a gastrointestinal (GI) system, a distal portion of the catheter including one or more exit ports communicating with a first lumen for administering a fluid. A deformable portion of the catheter being formed so that, in a first state, the deformable portion assumes a first shape suitable for guiding the catheter to the target location and, in a second state, the deformable portion assumes a second shape in which the deformable portion curves to extend in at least one radial direction for engaging an inner wall of the target location to anchor the catheter in the target location while administering the fluid via the lumen.

In an embodiment, the device further includes a guidewire sized and shaped to extend through the lumen of the catheter so that, when the guidewire is inserted within the deformable portion, the deformable portion assumes the first shape and, when the guidewire is removed, the deformable portion assumes the second shape.

In an embodiment, the guidewire comprises a rigidity greater than that of the deformable portion such that the deformable portion conforms to a shape of the guidewire when the guidewire is within the lumen.

In an embodiment, the second shape of the deformable portion comprises a helical coil or a spiral coil, the second shape being a natural shape of the deformable portion and inserting the guidewire within the deformable portion straightens the deformable portion into the first shape.

In an embodiment, the device further includes an electroactive polymer (EAP) incorporated into the catheter. The catheter transitions between the first and second states when a voltage is applied to the EAP or when the voltage is removed from the EAP.

In an embodiment, the device further includes a pull wire extending from a proximal end outside the human body through the lumen of the catheter to a distal end anchored to the catheter at or near the distal end of the catheter. The catheter includes a slit extending along the deformable portion such that, when tension is applied to the pull wire, the deformable portion curves as a distal portion of the pull wire translates through the slit to an exterior of the catheter to transition the deformable portion into the second state.

In an embodiment, the deformable portion comprises a series of slits extending at least transversely across the catheter, each slit forming a section of the catheter distal to the slit. The second shape comprises a spiral coil such that, when the deformable portion transitions into the second shape, proximal ends of the sections protrude to form barbs for engaging the inner wall of the target location.

In an embodiment, the deformable portion comprises a micropatterned surface for adhering to the inner wall of the target location.

In an embodiment, the deformable portion comprises multiple longitudinal slits forming multiple longitudinal sections. The device further includes an inner member extending through the lumen of the catheter, a distal end of the inner member fixed to the catheter distal to the deformable portion. Pulling the inner member forces the distal end of the catheter proximally such that the second shape comprises a radial expansion of the sections into arms for engaging the inner wall of the target location.

In an embodiment, the device further includes a film covering the deformable portion in the second shape to fully retain the fluid discharged via the deformable portion or a partial film covering a distal part of the deformable portion to partially retain the fluid.

In an embodiment, the catheter comprises a further deformable portion comprising multiple longitudinal slits forming multiple longitudinal sections. The deformable portion comprises a greater number of slits and sections than the further deformable portion. When the inner member is pulled, the deformable portion and the further deformable portion deploy in sequence.

In an embodiment, the catheter comprises a further deformable portion comprising multiple longitudinal slits forming multiple longitudinal sections. The deformable portion is longer than the further deformable portion. When the inner member is pulled, the deformable portion and the further deformable portion deploy in sequence.

In an embodiment, the deformable portion comprises multiple slits in a helical spiral pattern forming multiple sections. The device further includes an inner member extending through the lumen of the catheter, a distal end of the inner member fixed to the catheter distal to the deformable portion. Rotating the inner member forces the distal end of the catheter to rotate such that the second shape comprises a radial expansion of the sections into arms for engaging the inner wall of the target location.

In an embodiment, the device further includes an auxetic material within the deformable portion that expands to expand the deformable portion into the second shape by an elongation or extension applied to the auxetic material.

In an embodiment, the second shape comprises a scrunched shape creating peaks for engaging the inner wall of a small bowel and valleys for receiving folds of tissue to retain the catheter in position.

In addition, the present disclosure relates to a method for administering a fluid to a patient anatomy. The method includes guiding a flexible catheter to a target location within a gastrointestinal (GI) system, the catheter including a lumen extending therethrough, the catheter being sized and shaped to extend from a proximal end outside a human body to a distal end, a distal portion of the catheter including one or more exit ports communicating with a first lumen for administering a fluid, a deformable portion of the catheter being formed so that, in a first state, the deformable portion assumes a first shape suitable for guiding the catheter to the target location; and transitioning the catheter into a second state wherein the deformable portion assumes a second shape in which the deformable portion curves to extend in at least one radial direction for engaging an inner wall of the target location to anchor the catheter in the target location while administering the fluid via the lumen.

In an embodiment, the deformable portion assumes the first shape when a guidewire is extended through the lumen of the catheter and, when the guidewire is removed, the deformable portion assumes the second shape, the guidewire comprising a rigidity greater than that of the deformable portion such that the deformable portion conforms to a shape of the guidewire when the guidewire is within the lumen; or wherein an electroactive polymer (EAP) is incorporated into the catheter, wherein the catheter transitions between the first and second states when a voltage is applied to the EAP or when the voltage is removed from the EAP; or wherein a pull wire extends from a proximal end outside the human body through the lumen of the catheter to a distal end anchored to the catheter at or near the distal end of the catheter, wherein the catheter includes a slit extending along the deformable portion such that, when tension is applied to the pull wire, the deformable portion curves as a distal portion of the pull wire translates through the slit to an exterior of the catheter to transition the deformable portion into the second state.

In an embodiment, the deformable portion comprises a series of slits extending at least transversely across the catheter, each slit forming a section of the catheter distal to the slit and wherein the second shape comprises a spiral coil such that, when the deformable portion transitions into the second shape, proximal ends of the sections protrude to form barbs for engaging the inner wall of the target location; or wherein the deformable portion comprises a micropatterned surface for adhering to the inner wall of the target location.

In an embodiment, the deformable portion comprises multiple longitudinal slits forming multiple longitudinal sections, an inner member extends through the lumen of the catheter, a distal end of the inner member fixed to the catheter distal to the deformable portion, wherein pulling the inner member forces the distal end of the catheter proximally such that the second shape comprises a radial expansion of the sections into arms for engaging the inner wall of the target location.

In an embodiment, a film covers the deformable portion in the second shape to fully retain the fluid discharged via the deformable portion or a partial film covers a distal part of the deformable portion to partially retain the fluid.

### BRIEF DESCRIPTION

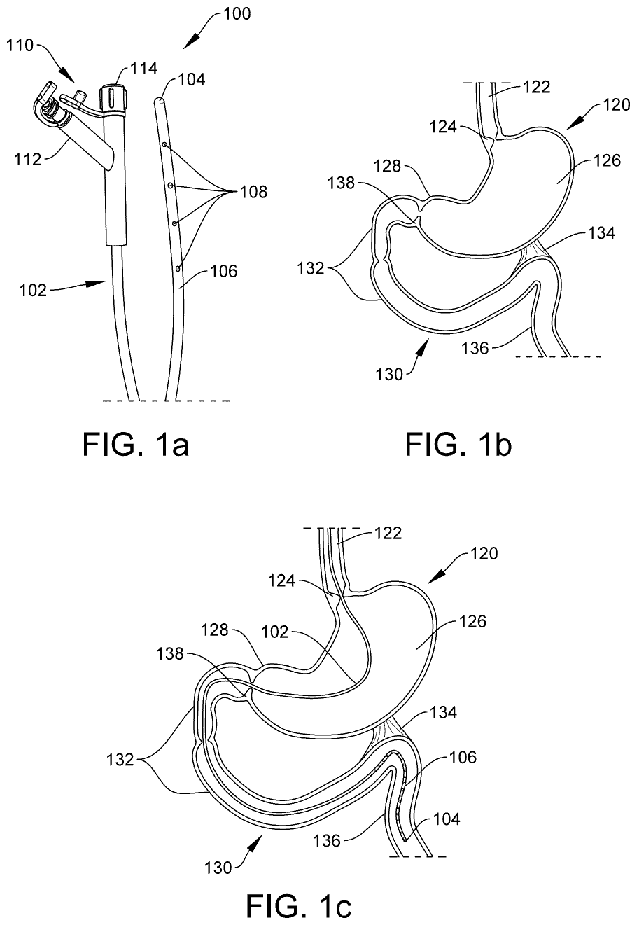

FIG. 1a shows a nasojejunal (NJ) tube device according to one example.

FIG. 1b shows a patient anatomy prior to inserting a NJ tube according to one example.

FIG. 1c shows the catheter of the NJ tube device of FIG. 1a placed in the patient anatomy of FIG. 1b according to one example.

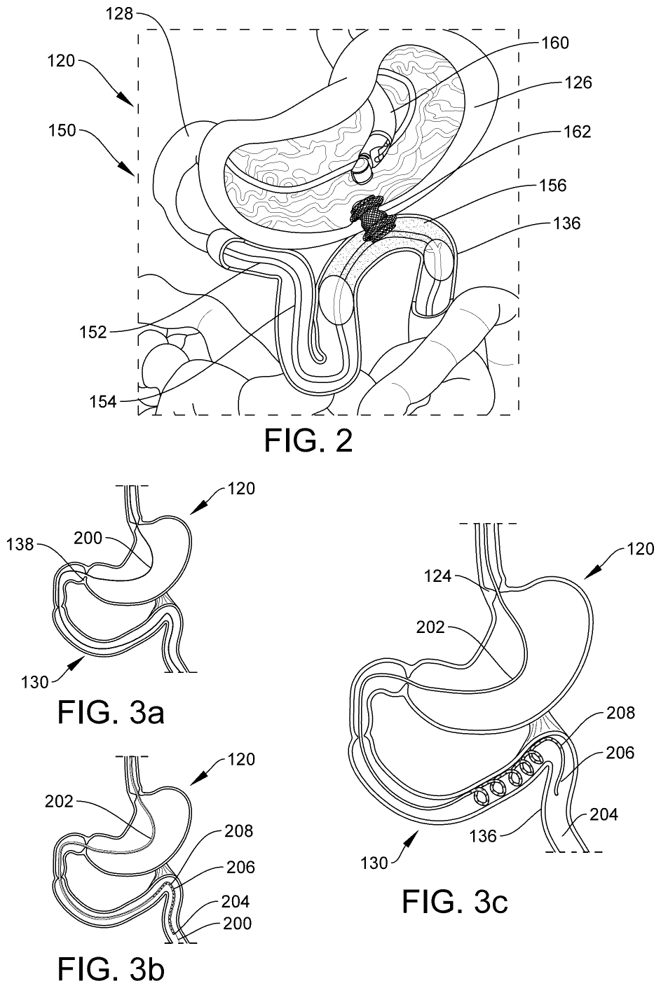

FIG. 2 shows a EUS-GE system for placing a gastro-jejunal stent in the anatomy of FIG. 1b according to one example.

FIG. 3a shows the patient anatomy of FIG. 1b with a guidewire advanced into the small bowel according to various exemplary embodiments.

FIG. 3b shows a catheter in an insertion state (first shape) advanced over the guidewire of FIG. 3a according to a first example of various exemplary embodiments.

FIG. 3c shows the catheter of FIG. 3b transitioned into an expanded state (second shape) when the guidewire is removed according to the first exemplary embodiment.

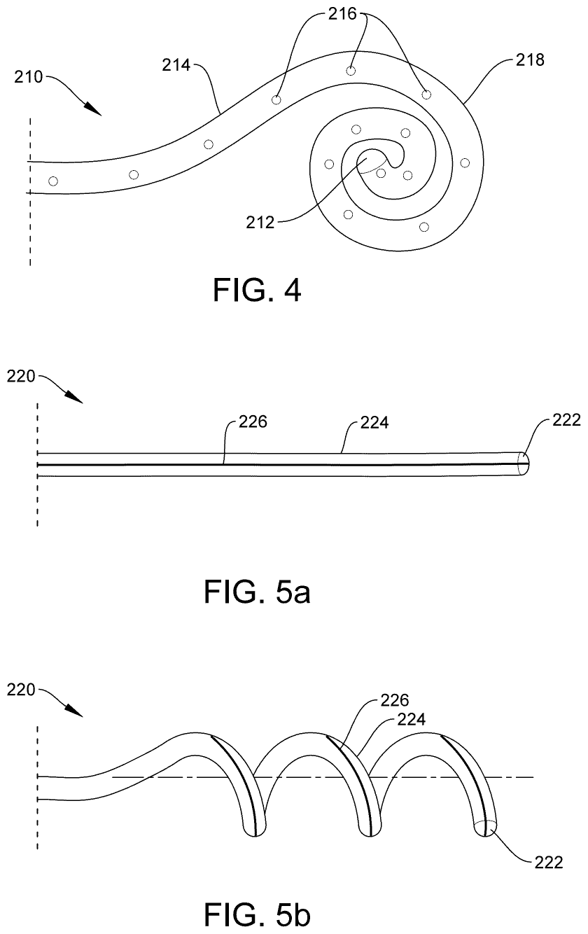

FIG. 4 shows a catheter in an expanded state (second shape) according to a second example of these exemplary embodiments.

FIG. 5a shows a catheter comprising an electro-active polymer (EAP) in an insertion state (first shape) when a voltage is applied thereto according to a third example of these exemplary embodiments.

FIG. 5b shows the catheter of FIG. 5a in an expanded state (second shape) when the voltage is not applied to the EAP according to the third exemplary embodiment.

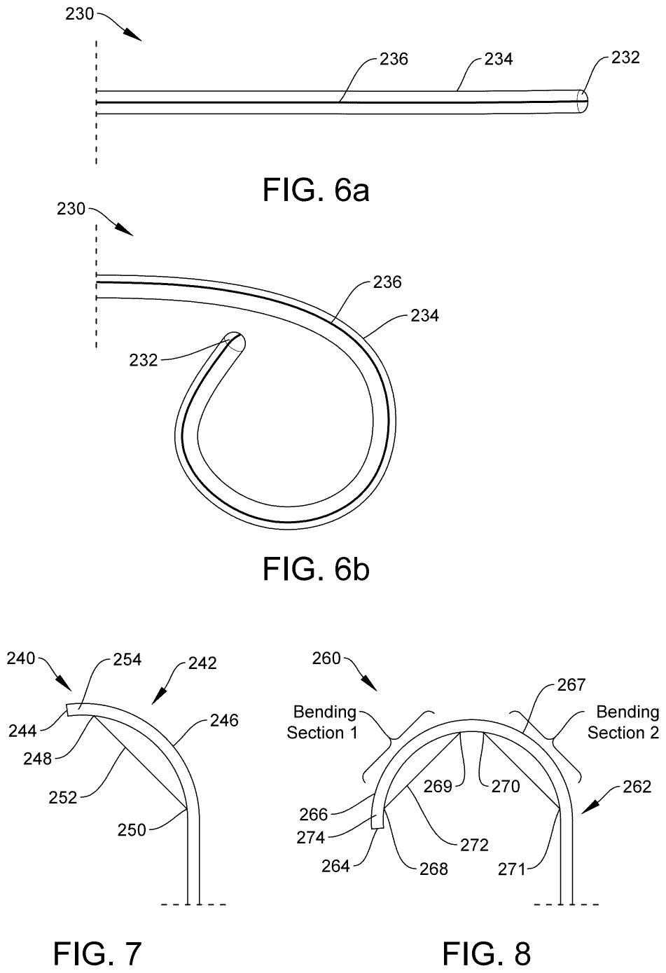

FIG. 6a shows a catheter comprising an EAP in an insertion state (first shape) when a voltage is applied thereto according to a fourth example of these exemplary embodiments.

FIG. 6b shows the catheter of FIG. 6a in an expanded state (second shape) when the voltage is not applied to the EAP according to the fourth exemplary embodiment.

FIG. 7 shows a catheter device comprising a pull wire for transitioning a catheter into an expanded state (second shape) according to a fifth example of these exemplary embodiments.

FIG. 8 shows a catheter device comprising a pull wire for transitioning a catheter into an expanded state (second shape) according to a sixth example of these exemplary embodiments.

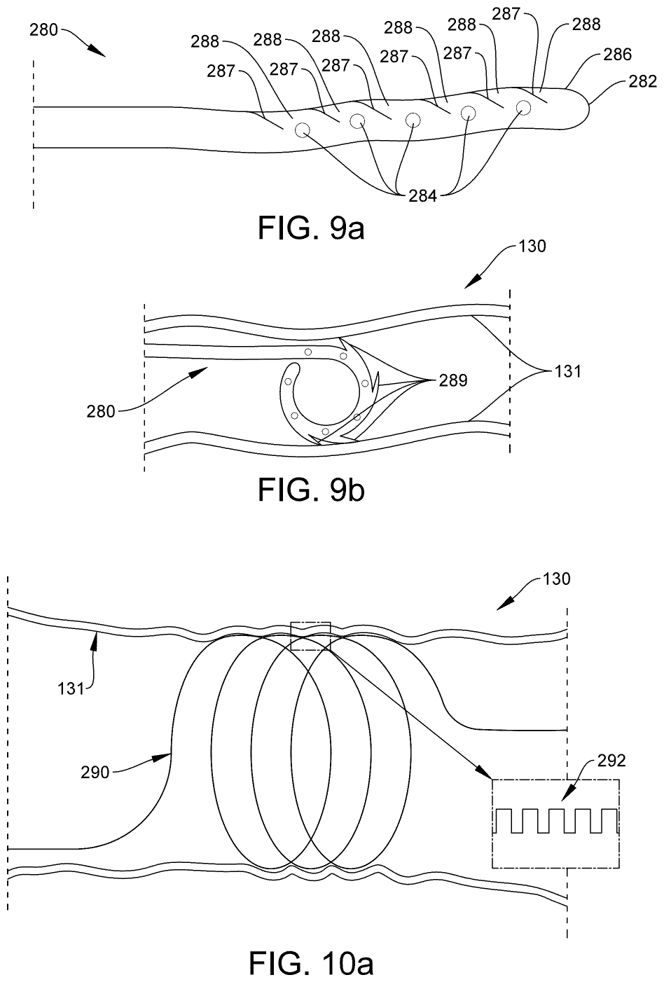

FIG. 9a shows a catheter comprising slits in the walls of the catheter forming sections in an insertion state (first shape) according to a seventh example of these exemplary embodiments.

FIG. 9b shows the catheter of FIG. 9a in an expanded state (second shape) wherein barbs are formed by the sections according to the seventh exemplary embodiment.

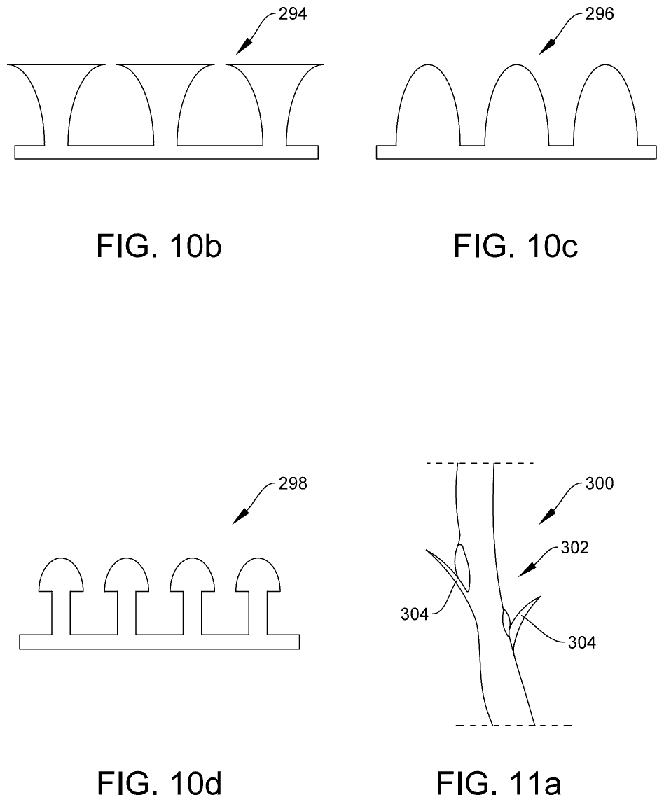

FIG. 10a shows a catheter comprising a first micropatterned surface according to an eighth example of these exemplary embodiments.

FIG. 10b shows a second micropatterned surface according to a ninth example of these exemplary embodiments.

FIG. 10c shows a third micropatterned surface according to a tenth example of these exemplary embodiments.

FIG. 10d shows a fourth micropatterned surface according to an eleventh example of these exemplary embodiments.

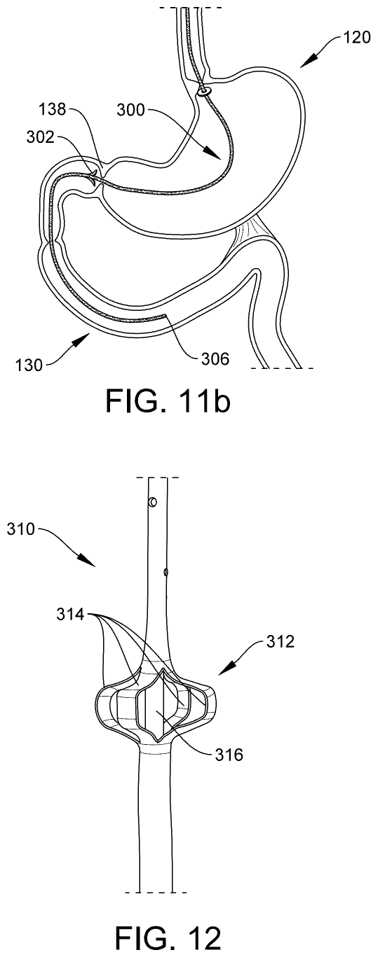

FIG. 11a shows a catheter comprising a barbed portion according to a twelfth example of these exemplary embodiments.

FIG. 11b shows the catheter of FIG. 11a deployed in the patient anatomy of FIG. 1b according to the twelfth exemplary embodiment.

FIG. 12 shows a catheter comprising a Malecot-type anchor according to one example.

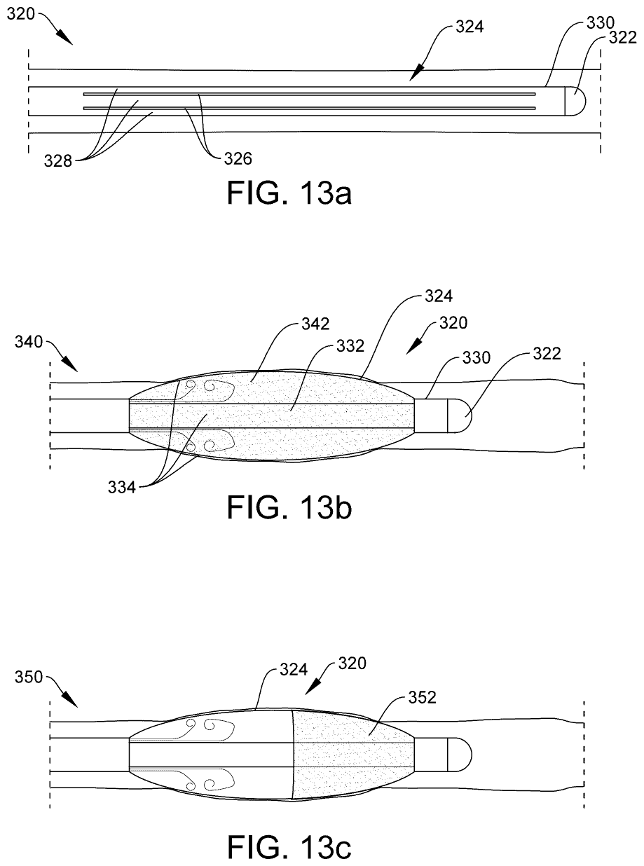

FIG. 13a shows a catheter comprising Malecot-type features in an insertion state (first shape) according to a thirteenth example of these exemplary embodiments.

FIG. 13b shows a catheter comprising the catheter of FIG. 13a and further including a film cover in an expanded state (second shape) according to a fourteenth example of these exemplary embodiments.

FIG. 13c shows a catheter comprising the catheter of FIG. 13b and further including a partial film cover in an expanded state (second shape) according to a fifteenth example of these exemplary embodiments.

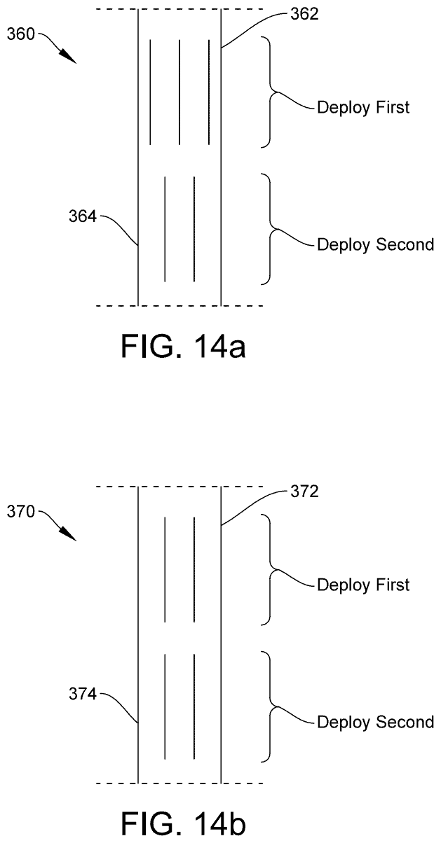

FIG. 14a shows a catheter comprising multiple Malecot-type features according to a sixteenth example of these exemplary embodiments.

FIG. 14b shows a catheter comprising multiple Malecot-type features according to a seventeenth example of these exemplary embodiments.

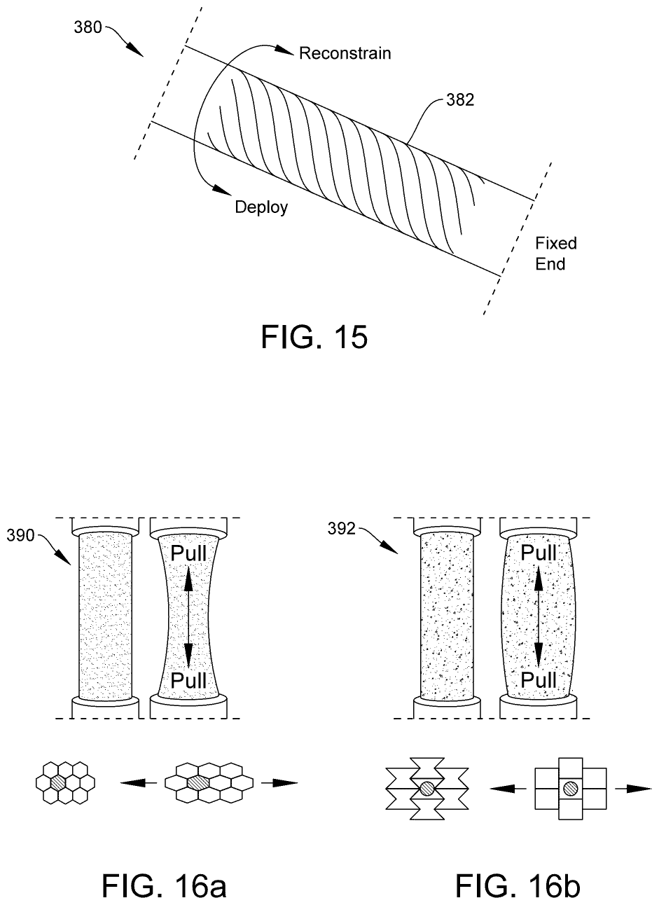

FIG. 15 shows a catheter comprising spiral cut Malecot-type features according to an eighteenth example of these exemplary embodiments.

FIG. 16a shows a conventional material according to one example.

FIG. 16b shows an auxetic material. according to one example.

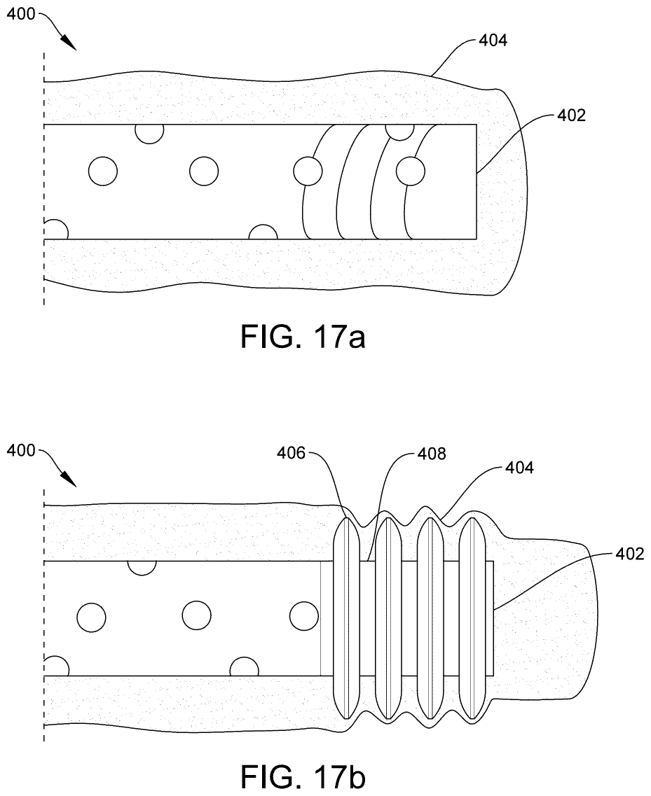

FIG. 17a shows a catheter comprising a deformable portion in an insertion state (first shape) according to a nineteenth example of these exemplary embodiments.

FIG. 17b shows the catheter of FIG. 17a in an expanded state (second shape) according to the nineteenth exemplary embodiment.

### DETAILED DESCRIPTION

The present disclosure may be further understood with reference to the following description and the appended drawings, wherein like elements are referred to with the same reference numerals. The present disclosure relates to nasojejunal (NJ) tubes and/or catheters including features for retention at a target location in the gastrointestinal (GI) tract. In particular, the exemplary catheters are configured to transition from a first shape or state suitable for advancing the catheter through the GI tract to a second shape or state in which the catheter expands in two or three dimensions to engage an inner wall of the small bowel, e.g., the duodenum or the jejunum.

Pyloric stenosis involves a narrowing of the gastric outlet (pylorus) which may cause difficulties in passing food from the stomach to the small bowel. This may lead to gastric outlet obstruction (stricture) reducing or completely impeding the ability of the patient to digest food and absorb nutrients. Similar obstructions may also occur in the small bowel, e.g., the duodenum. As indicated above, there are several currently employed treatments for this problem, including placing a nasojejunal (NJ) feeding tube in the small bowel. The NJ tube is fed down the esophagus into the stomach, e.g., via endoscope or guidewire, and guided through the stricture so that nutrients may be provided to the small bowel, e.g., the jejunum, using the tube to bypass the stricture. The NJ tube typically comprises holes along a length of a portion of the tube near the distal end of the tube so that this portion, when positioned as desired, discharges nutrients from the tube into the target anatomical structure (e.g., small bowel) via the holes.

FIG. 1a shows a nasojejunal (NJ) tube device 100 according to one example. The device includes a catheter 102 (e.g., tube) extending from a proximal end (not shown) to a distal end 104 with an atraumatic distal tip configured to be advanced through the gastrointestinal (GI) tract. The catheter 102 of this example comprises a single lumen or channel. The catheter 102 has a distal portion 106 near or adjacent to the distal end 104 comprising holes 108 configured to discharge a fluid administered through the lumen of the catheter 102 into a target anatomical structure. In short, the distal portion 106 of the catheter 102 extends along a portion of the catheter 102 that is to be positioned within a target anatomical area into which fluids are to be supplied via the catheter. The catheter 102 of this embodiment is configured to be guided through the GI tract to a position in which the distal portion 106 is located in the small bowel, e.g., the jejunum.

The NJ tube device 100 further comprises a handle 110 including a port 112 in fluid communication with and configured to access the lumen of the catheter, e.g., to administer the fluid from the port 112 to the target anatomical structure via the lumen of the catheter 102. The catheter 102 is configured (sized and shaped) to be advanced longitudinally through a channel of the handle 110 as the catheter 102 is guided to the target location. The proximal end of the catheter 102 is fixed to a cap 114 configured to interface with the handle 110 when the catheter 102 is fully extended distally relative to the handle 110.

FIG. 1b shows patient anatomy 120 prior to inserting a NJ tube according to one example. The anatomy 120 comprises a portion of the GI tract including the esophagus 122, the stomach 126 and the small bowel 130 (small intestine). The small bowel 130 includes the duodenum 132, the jejunum 136, and the ileum (not shown). As would be understood by those skilled in the art, the esophogeal sphincter 124 corresponds to the end of the esophagus 122 and the start of the stomach 126, the pylorus 128 corresponds to the end of the stomach 126 and the start of the duodenum 132 and the ligament of Treitz 134 is a ligament supporting the small bowel and corresponds to the end of the duodenum 132 and the start of the jejunum 136. Pyloric stenosis refers to a narrowing of the pylorus 128 that can create a stricture 138 preventing or making difficult the passage of food from the stomach 126 to the small bowel 130.

FIG. 1c shows the catheter 102 of the NJ tube device 100 of FIG. 1a placed in the anatomy 120 of FIG. 1b according to one example. The catheter 102 of the NJ tube device 100 is configured to be passed down the esophagus 122, through the stomach 126, past the stricture 138 (e.g., the pylorus 128 which is narrowed) and into the small bowel 130 under visualization (e.g., via endoscope) by using a guidewire, and/or by other methods. The catheter 102 is configured to be passed through the duodenum 132 past the ligament of Treitz 134 to the jejunum 136 so that the distal portion 106 comprising the holes 108 is positioned at least partially in the jejunum 136. In other cases, the catheter 102 can be placed further proximally in the duodenum. Once the catheter 102 has been placed at the desired location, fluids can be administered via the handle 110 and discharged into the jejunum 136 via the holes 108.

In this example, the NJ tube device 100 is for feeding purposes and the fluids administered by the tube comprise nutrients for the patient. In other examples, the fluid can comprise contrast or other therapeutic fluids, e.g., medicines, fluids configured to enhance visualization for assisting endoscopic ultrasound guided gastroenterostomy (EUS-GE) procedures, etc.

Another method for treating a patient with pyloric stenosis includes the creation of a surgical gastro-jejunostomy bypass bridging the stomach and the jejunum to bypass the area of the stricture. NJ tubes can be used to assist with EUS-GE procedures by instilling contrast or another fluid into the jejunum to enhance visualization of the anatomy. In one example, contrast is provided to show a target to an operating physician, e.g., for a procedure including piercing the stomach and the jejunum and placing a stent (e.g., AXIOS stent) therebetween to bypass the pylorus. It is noted that EUS-GE procedures may alternatively target the duodenum and any reference to the jejunum as the target location is for exemplary purposes only.

FIG. 2 shows an EUS-GE system 150 for placing a gastro-jejunal stent 162 in the anatomy 120 of FIG. 1b according to one example. The EUS-GE system 150 comprises a NJ tube device 152 including a catheter 154 that is passed into the jejunum 136 so that fluid can be discharged into the jejunum 136. In this example, the fluid comprises contrast fluid 156 to help the operating physician locate the target location.

The EUS-GE system 150 of this example further comprises an endoscope 160 for deploying the stent 162. A piercing element (not shown) can be deployed via the endoscope 160 to pierce the stomach 126 and the jejunum 136 as would be understood by those skilled in the art. The stent 162 is placed to bridge the stomach 126 and the jejunum 136 bypassing the pylorus 128.

One issue with NJ tubes is the tube drifting out of position during use such that fluids discharged by the tube may be provided to an unintended location. In one example, the NJ tube can migrate proximally during use. In feeding tube applications, this proximal drift can cause nutrients to be provided further up in the small bowel, or even in the stomach proximal to the stricture, which would cause issues with digestion.

In EUS-GE applications, the drift of the NJ tube can cause fluids (e.g., contrast fluid) to be instilled away from an intended target area. With regard to the gastro-jejunostomy procedure described in FIG. 2, without proper visualization, stents for bypassing the pylorus may be misdeployed. In some examples, the stent may be deployed in a hole created in the stomach but without capturing the jejunum (which may or may not have been pierced by the piercing element); similarly, the stent can be deployed in the hole created in the jejunum but without capturing the stomach; or the stent may be deployed to bridge the stomach and the colon. Accordingly, there is a need for improved devices and mechanisms to retain a tube in a desired location in the GI tract.

According to various exemplary embodiments, systems, devices and methods are described for retaining a catheter, e.g., a nasojejunal tube, at a desired location in the small bowel. The catheter can be retained at a position in the small bowel such that the holes for discharging a fluid are positioned in a desired location, e.g., the jejunum. Some of the devices described herein include a catheter intended for use as a feeding tube, e.g., for extended use (weeks, months, etc.), and some of the devices described herein include catheters intended for providing fluid during EUS-GE procedures, as will be described below. However, it is noted that many of the described embodiments are equally applicable for either purpose, as described in detail below.

The terms “NJ tube” and/or “catheter” are used herein to describe a tube configured for deployment within the GI tract for administering a fluid, e.g., for feeding, a therapeutic fluid or a fluid applied to facilitate a procedure such as EUS-GE. The catheters according to the present embodiments generally have a single lumen or channel for passing a fluid through the catheter from a proximal end to a distal portion from which the fluid can be discharged through holes in the wall of the catheter. In some cases, the catheter may not have holes in its wall but rather may have an open distal tip for discharging the fluid. Alternatively, a catheter may have both a series of holes located along a length of a distal portion thereof and an open distal end.

Those skilled in the art understand that a simple catheter can comprise a length of tube, e.g., formed of a material such as a plastic and/or polymer (e.g., PVC, PTFE, etc.) with a desired flexibility and/or rigidity, that may assume a substantially straight (longitudinal) or slightly curved shape when no external forces are applied at any location along the length of the tube. However, the simple catheter is typically sufficiently flexible such that during deployment the catheter can bend or curve under external forces imposed by, e.g., an operating physician at the proximal end of the catheter; by contacting an inner wall of an endoscope channel; by contacting inner walls of organs along the GI tract; or by passing the catheter over a guidewire. Accordingly, the simple catheter can assume many variations of three-dimensional shapes due to its flexibility and various external forces imposed at various locations along its length allowing the catheter to extend along a tortuous path. However, those skilled in the art understand that the ability of the catheter to curve or bend is generally limited and such bending typically proceeds in a snake-like manner, e.g., as body lumens are traversed. Those skilled in the art will ascertain that a simple catheter generally does not expand in shape, for example, radially, relative to its circular cross-section along its length.

In various exemplary embodiments described herein, a catheter can be deformable and transition between a first state or shape (e.g., insertion shape/state or contracted shape/state) into a second state or shape (e.g., deployment shape/state or expanded shape/state). The catheter in the second state can be sized, shaped and otherwise designed for contacting an inner wall of the small bowel for retaining the catheter therein. Relative to a simple catheter, which can deform during deployment (e.g., as body lumens are traversed as described above), the catheters according to these exemplary embodiments are configured to deform independently from the external forces that are typically applied when placing a catheter.

In one aspect of these exemplary embodiments, a catheter is formed so that its natural shape, e.g., a shape of the catheter when it is under no external forces and/or under a natural bias, has a portion that is expanded relative to the remainder of the tube. In other words, the second shape suitable for retaining the catheter is the natural shape of the catheter when at rest. It should be understood that this portion of the catheter is considered to be expanded relative to the size and shape of a length of a remaining portion of the tube. The remainder of the tube (excluding the portion configured for expansion) can be shaped as a typical length of tube.

In these embodiments, a guidewire is inserted through the lumen of the catheter to apply a force internal to the catheter that causes the catheter to change shape, e.g., to assume the shape of a simple tube or catheter. In other words, the first shape suitable for advancing the catheter through the GI tract comprises a deformed shape, relative to its natural shape. The guidewire has a rigidity greater than that of the catheter such that, when the guidewire is inserted through the lumen of the catheter (and/or when the catheter is advanced over a previously placed guidewire), the portion of the catheter that is naturally expanded is forced to assume the shape of the guidewire. The catheter can be fed over the guidewire (in the first shape) to a target location within the small bowel. The guidewire can then be withdrawn proximally relative to the catheter so that the expandable portion can assume its natural shape (second shape) within the small bowel.

The catheter assumes the first shape with the guidewire inserted. The first shape may be referred to herein as a first state, an insertion state, a contracted shape, etc. Those skilled in the art understand that the catheter is flexible, such that a fully described “shape” of the catheter during use may include bends imposed by e.g., a patient anatomy through which the catheter is extended so that the distal end can reach a target site within the body. The “shape” referred to in the exemplary embodiments can be considered from a cross-sectional perspective, e.g., relative to a typical cross section of a tube. In other words, with the guidewire inserted (in the first shape), the catheter is in a substantially tube-like longitudinal shape such that the catheter can be guided to a target location in a typical manner, e.g., without excessive contact with the inner walls of the GI tract such that the catheter cannot effectively traverse the GI tract.

When the guidewire is removed, a portion of the catheter is then transitioned into a second shape under its natural bias. The second shape can include some aspect of expansion relative to the contracted shape such that the size of the portion of the catheter is increased, e.g., in two or three dimensions, to be described below. The second shape may be referred to herein as an expanded shape, a second state, a deployment state, etc. In some embodiments, the second shape comprises a helical coil. In other embodiments, the second shape comprises a spiral coil. Those skilled in the art will understand that other types of shapes may be configured for the expanded shape.

FIG. 3a shows the anatomy 120 of FIG. 1b with a guidewire 200 advanced into the small bowel according to various exemplary embodiments; FIG. 3b shows a catheter 202 in an insertion state (first shape) advanced over the guidewire 200 of FIG. 3a according to a first example of various exemplary embodiments; FIG. 3c shows the catheter 202 of FIG. 3b transitioned into an expanded state (second shape) when the guidewire 200 is removed according to the first exemplary embodiment.

As shown in FIG. 3a, the anatomy 120 comprises a portion of the GI tract including the esophagus, the stomach, and the small bowel, similar to the preceding figures. In this exemplary procedure, the guidewire 200 is introduced in the esophagus, passed through the stomach past the stricture 138 and placed such that a distal end thereof is within the small bowel 130. It should be understood that the guidewire 200 can be extended to a location within the small bowel 130 such that the catheter 202 can be passed over the guidewire 200 to a desired location within the small bowel 130, to be described below.

As shown in FIG. 3b, the catheter 202 is passed over the guidewire 200. The catheter 202 extends from a proximal end (not shown) to a distal end 204 with an atraumatic distal tip. Adjacent to the distal end 204 is a distal portion 206 comprising a plurality of holes (not labeled) configured to discharge fluid passed through the lumen of the catheter 202. The catheter 202 further comprises a deformable portion 208 configured to transition between a first shape and a second shape. When the guidewire 200 is within the deformable portion 208 the catheter 202 assumes the first shape. The first shape, e.g., an insertion state, is substantially similar to that of a simple tube or catheter (e.g., “straight”) such that the catheter 202 can be passed through the GI tract.

In this example, the deformable portion 208 comprises a length of the catheter 202 adjacent to but not including the distal end 204. However, it should be understood that in other embodiments the deformable portion 208 can extend to the distal end 204 or can be located further proximally along the catheter 202. The catheter 202 is passed over the guidewire 200 through the GI tract such that the deformable portion 208 is at a desired location within the small bowel, to be explained in further detail below.

As shown in FIG. 3c, the guidewire 200 is removed and the deformable portion 208 of the catheter 202 transitions into the second shape (or expanded shape). In this example, the expanded shape is a helical coil, e.g., the deformable portion 208 rotates along its length about a longitudinal axis of the catheter 202. Those skilled in the art will understand that the transition of the catheter to the shape of a helical coil is considered a radial expansion and/or increased volume, relative to the cross section of the catheter 202 in the first shape as this helical shape brings the outer surface of the deformable portion 208 into contact with the inner walls of the small bowel. In the second shape, the contact between the deformable portion 208 and the small bowel holds the catheter 202 in a relatively fixed position within the small bowel.

The deformable portion 208 may coincide completely, substantially, or in part with the distal portion 206 of the catheter 202 comprising the holes for discharging fluid from the catheter 202. In other words, the holes for discharging the fluid may be found completely on the deformable portion 208 and/or on either one or both the proximal and distal sides of the deformable portion 208. The location of the deformable portion 208 relative to the distal portion 206 including the holes may depend on a desired location for discharging the fluid relative to a desired location for contacting the small bowel to retain the position of the catheter 202. In this example, the catheter 202 is configured so that the deformable portion 208 substantially coincides with the distal portion 206 comprising the holes. The deformable portion 208 can contact the inner wall of the duodenum adjacent to the ligament of Treitz when the catheter 202 is transitioned into the expanded shape and the fluid can be discharged from the holes in a similar location.

It is noted that, in the example of FIGS. 3a-c, the catheter 202 may be used as a feeding tube and the deployment location of the deformable portion 208 and/or distal portion 206 may be selected accordingly to provide nutrients that will be digested at a proper location, e.g., at the beginning of the jejunum. In these applications, the catheter 202 can remain in the expanded shape for retention in the small bowel over an extended period of time (weeks, months, etc.). However, it should be understood that a similar catheter may be deployed so that the deformable portion 208 and/or the distal portion 206 is placed further distally, e.g., within the jejunum, so that fluid can be applied at a further distal location, e.g., to assist an EUS-GE procedure.

The holes for discharging the fluid can be oriented so that, when the catheter 202 transitions into the helically coiled shape, the holes are directed toward an interior (e.g., toward a centerline) of the helix, or in some other direction that is not facing the inner wall of the small bowel, thus allowing a fluid discharged from the holes to pass into the small bowel without obstruction by the inner wall of the small bowel. As would be understood by those skilled in the art, the holes for discharging fluid of this embodiment are formed on portions of the outer surface of the deformable portion of the catheter 202 which, when in the second shape, face radially inward (i.e., toward a central axis of the helix) so that the holes are not obstructed by contact with the tissue of the inner wall of the duodenum. In addition, it is noted that these holes will preferably be positioned in all embodiments to avoid obstruction from the tissue surrounding the catheter.

The guidewire 200 can be reinserted to transition the catheter 202 back into the insertion shape to disengage the deformable portion 208 from the small bowel to remove or reposition the catheter 202. The catheter 202 can be withdrawn proximally over the guidewire 200, which may then be removed, or both the catheter 202 and guidewire 200 can be removed together.

In other embodiments, the second shape of the deformable catheter may comprise a coil about an axis transverse to the longitudinal axis of the catheter. In other words, the catheter can twist back on itself in a single plane to form a spiral or fiddlehead shape. It should be understood that, in these embodiments, the length of the deformable portion will dictate the degree to which the deformable portion rotates, e.g., a number of rotations composing the spiral. In these embodiments, the deformable portion of the catheter comprises a length extending from and including the distal end of the catheter, such that withdrawing the guidewire allows the deformable portion to wrap into the second shape. The coiled shape can be considered to have an expanded cross section (relative to a cross section of a simple catheter) and/or define a larger volume relative to a typical length of the tube.

FIG. 4 shows a catheter 210 in an expanded state (second shape) according to a second example of these exemplary embodiments. The catheter 210 extends from a proximal end (not shown) to a distal end 212 with an atraumatic distal tip. Adjacent to the distal end 212 is a distal portion 214 comprising holes 216 for discharging a fluid passed through the lumen of the catheter 210. The catheter 210 further comprises a deformable portion 218 configured to transition between a first shape and a second shape. The first shape may be substantially similar to the first shape of the catheter 202 of the first exemplary embodiment described with regard to FIG. 3b when a guidewire (not shown) is within the deformable portion 218 of the catheter 210.

In this example, the deformable portion 218 comprises a length of the catheter 210 extending proximally from and including the distal end 212. As shown in FIG. 4, when the guidewire is removed, the deformable portion 218 of the catheter 210 transitions into the second shape or expanded state under its natural bias. In this example, the expanded shape is a spiral or fiddlehead coil with the distal end 212 at the center thereof. It will be understood by those skilled in the art that the degree of bending of the catheter 210 in the expanded shape may depend on the length of the deformable portion 218, e.g., the spiral coil may comprise a greater or lesser number of rotations. The spiral coil comprises a radial expansion and/or increased volume, relative to the cross section of the catheter 210 in the first shape, such that the radially outer surface of the coiled deformable portion 218 may be brought into contact with the inner walls of the small bowel causing the catheter 210 anchoring the catheter 210 at a desired fixed position within the small bowel.

In this example, the deformable portion 218 includes at least part of the distal portion 214 including the holes 216. If the catheter 210 is used as a feeding tube it may be deployed with the deformable portion 218 located at a desired location to which nutrients are to be supplied to the patient, e.g., the duodenum and/or jejunum, that can be administered via the holes 216 found along the deformable portion 218. If the catheter 210 is used for EUS-GE assistance, the deployment location may be positioned further into the jejunum. The holes 216 can be oriented so that, when the catheter 210 transitions into the spiral coiled shape, the holes 216 are directed parallel to the axis of rotation of the deformable portion 218, as shown in FIG. 4, thus allowing fluid discharged from the holes 216 to pass into the small bowel without obstruction by the coiled surfaces of the catheter 210.

In another aspect of these exemplary embodiments, the catheter can incorporate an electro-active polymer (EAP). EAPs are similar to conventional piezoelectric crystals in that they undergo a change in length or shape when an electric voltage is applied. However, since EAPs are primarily composed of polymers, the resulting material is flexible and better suited to use inside the body.

In these embodiments, the catheter can include a strip of EAP material embedded within the wall of the catheter, which is otherwise formed of a flexible material (e.g., plastic and/or polymer) as described above. The EAP strip is configured so that, when subjected to a predetermined applied voltage, the EAP strip assumes a first shape. The physical force imposed by the EAP strip under voltage to assume the first shape overcomes internal forces of the material composing the remainder of the catheter, such that the portion of the catheter carrying the EAP strip assumes a shape similar to that of the EAP strip. When the voltage is removed, the EAP strip becomes flexible and no longer exerts a deforming force on the catheter. Thus, the EAP strip assumes a second shape which is generally the natural shape of the catheter.

It may be considered that the following two options are available for designing a catheter incorporating an EAP according to the present embodiments, including: 1) the catheter assumes an expanded shape at rest (when the voltage is removed) (second shape) and assumes a contracted shape (e.g., “straight”) (first shape) when the voltage is applied or 2) the catheter assumes a relatively straight shape at rest (first shape) and assumes an expanded shape (second shape) when the voltage is applied. Either option may be preferable in various scenarios. However, the first option may be preferred in many cases so that the voltage is temporarily applied to straighten the catheter for the purpose of placement. After being placed, the voltage may be removed. The second option would require the voltage to be constantly applied while the tube is in use at a target location.

The EAP may be disposed asymmetrically around the circumference of the distal portion of the catheter, such that activation by voltage induces a shape change that re-directs the longitudinal axis of the NJ tube. In some embodiments, the EAP may be disposed along the outermost (longest) edge of a distal portion of an NJ tube. When voltage is applied, the EAP strip contracts, straightening the coil. In some embodiments, the second shape comprises a helical coil and, in other embodiments, the second shape comprises a spiral coil, similar to the respective catheters 202, 210 of the first and second exemplary embodiments described in FIGS. 3c, 4. Those skilled in the art will understand that other types of shapes may be configured for the expanded shape.

FIG. 5a shows a catheter 220 comprising an electro-active polymer (EAP) in an insertion state (first shape) when a voltage is applied thereto according to a third example of these exemplary embodiments; FIG. 5b shows the catheter 220 of FIG. 5a in an expanded state (second shape) when the voltage is not applied to the EAP according to the third exemplary embodiment.

As shown in FIG. 5a, the catheter 220 assumes a contracted shape, e.g., straight shape typical of a simple catheter, when the voltage is applied. The catheter 220 extends from a proximal end (not shown) to a distal end 222 with an atraumatic distal tip. In this example, a distal portion comprising holes for discharging a fluid passed through the lumen of the catheter is included but not shown. However, it should be understood that the holes can be provided at locations similar to those as described above for the catheter 220 of FIGS. 3b-c or in any other desired arrangement. The catheter 220 comprises a deformable portion 224 configured to transition between a first shape and a second shape. In this example, the deformable portion 224 of the catheter 220 is deformed by the application and/or removal of a voltage to an EAP strip 226. The first shape, e.g., an insertion state, is substantially similar to that of a simple tube or catheter when the voltage is applied to the EAP strip 226 such that the catheter 202 can be passed through the GI tract.

In this example, the EAP strip 226 extends along a length of the catheter 220 proximally from and including the distal end 222 such that the deformable portion 224 of the catheter at least includes the length over which the EAP strip 226 extends. However, similar to the catheter 220 of FIGS. 3b-c, it should be understood that the EAP strip 226 and the deformable portion 224 may be located further proximally along the catheter 220. The catheter 220 in the first shape is passed through the GI tract such that the deformable portion 224 is at a desired location within the small bowel.

As shown in FIG. 5b, when the voltage is removed, the deformable portion 224 of the catheter 220 transitions to the second shape or expanded shape. In this example, the expanded shape is a helical coil similar to the catheter 220 of FIGS. 3b-c. In the second shape, contact between the deformable portion 224 and the small bowel anchors the catheter 220 in a relatively fixed position within the small bowel.

Similar to the preceding embodiments, the catheter 220 can be used as a feeding tube or to assist an EUS-GE procedure. The voltage can be reapplied to transition the catheter 220 back into the first shape to remove the catheter 220 from the patient anatomy.

FIG. 6a shows a catheter 230 comprising an EAP in an insertion state (first shape) when a voltage is applied thereto according to a fourth example of these exemplary embodiments; FIG. 6b shows the catheter 230 of FIG. 6a in an expanded state (second shape) when the voltage is not applied to the EAP according to the fourth exemplary embodiment.

As shown in FIG. 6a, the catheter 230 assumes a contracted shape when the voltage is applied, similar to the catheter 220 of FIGS. 5a. The catheter 230 extends from a proximal end (not shown) to a distal end 232 with an atraumatic distal tip and includes a distal portion comprising holes for discharging a fluid passed through the lumen of the catheter. The catheter 230 comprises a deformable portion 234 configured to transition between a first shape and a second shape by the application and/or removal of a voltage to an EAP strip 236. It should be understood that the holes can be provided at locations similar to those as described above for the catheter 210 of FIG. 4, e.g., along the deformable portion 234.

In this example, the EAP strip 236 extends along a length of the catheter 230 extending proximally from and including the distal end 232 such that the deformable portion 234 of the catheter includes at least this length. The catheter 230 in the first shape is passed through the GI tract such that the deformable portion 234 is at a desired location within the small bowel.

As shown in FIG. 6b, when the voltage is removed, the deformable portion 234 of the catheter 230 transitions to the second shape or expanded shape. In this example, the expanded shape is a spiral coil, similar to the catheter 210 of FIG. 4. It should be understood that a degree of bending and/or number of rotations composing the second shape can be varied. The second shape is selected so that contact between the deformable portion 234 and the small bowel anchors the catheter 230 in a relatively fixed position within the small bowel.

Similar to the preceding embodiments, the catheter 230 can be used as a feeding tube or to assist an EUS-GE procedure. To remove the catheter 230 from the patient, the voltage can be reapplied to transition the catheter 230 back into the first shape.

In another aspect of these exemplary embodiments, a catheter device incorporates a pull wire fixed to a catheter for transitioning the catheter between the insertion shape and the expanded shape. It may be considered that these embodiments relate to Tome-style devices. Tomes employ a wire that deploys from a single slit along the side of a catheter. As the wire is pulled it extends through the slit (outside the catheter) causing the catheter to bend. In other words, the distal end of the wire pulls the distal end of the catheter toward the proximal end of the slit, causing the catheter to bend while the pull wire travels in a straight (tensioned) line between the distal end of the slit and the proximal end of the slit, outside of (and not following the curvature of) the catheter. It is noted that this is different from Malecot-style devices, where the element in tension remains longitudinally centered, as will be described in detail further below with regard to further exemplary embodiments. The curved shape produced by Tome-style deployment may be utilized as an anti-migration feature in an NJ tube. Further, multiple curving sections may be chained in series to produce a larger final curved shape.

FIG. 7 shows a catheter device 240 comprising a pull wire 252 for transitioning a catheter 242 into an expanded state (second shape) according to a fifth example of these exemplary embodiments. The catheter 242 can assume an insertion state (first shape), e.g., straight shape typical of a simple catheter, when no tension is applied to the pull wire 252. The catheter 242 extends from a proximal end (not shown) to a distal end 244 with an atraumatic distal tip and includes a distal portion comprising holes (not shown) configured to discharge fluid passed through the lumen of the catheter. It should be understood that the holes can be provided at various locations starting at or adjacent to the distal end 244, similar to those described in the preceding embodiments. The catheter 242 comprises a deformable portion 246 adjacent to the distal end 244 configured to transition between a first shape and a second shape by the application and/or removal of tension to the pull wire 252.

In this example, the deformable portion 246 includes a single slit in its wall (in a plane that is not visible in FIG. 7) extending along its length. A distal end 248 of the slit provides an anchor position for a distal end 254 of the pull wire 252. A proximal end 250 of the slit provides a surface over which the pull wire 252 can be drawn when the deformable portion 246 begins to bend, to be explained further below. The pull wire 252 runs loosely within the lumen of the catheter from the anchor point at its distal end 254 to a proximal end (not shown) that can be tensioned by an operating physician. The catheter 242 in the first shape is passed through the GI tract such that the deformable portion 246 is at a desired location within the small bowel.

As shown in FIG. 7, when tension is applied to the pull wire 252, the deformable portion 246 of the catheter device 240 transitions into the second shape or expanded shape. Specifically, the slit in the catheter 242 allows the pull wire 252 to pass outside the lumen of the catheter 242 such that tensioning the pull wire 252 to apply a distal force at the anchor point near the distal end 248 of the slit, bends the catheter 242 along the portion comprising the slit (the deformable portion 246). The proximal end 250 of the slit restricts movement of the pull wire 252 in two dimensions (excluding a longitudinal axis) such that the pull wire 252 is drawn proximally over the proximal end 250 of the slit while remaining in contact with the proximal end 250 of the slit as tension is applied. In this example, the second shape is a substantially 90 degree bend.

However, it should be understood that different bend radii can be applied based on different applications of tension. In other words, applying increased tension to the pull wire 252 will bend the deformable portion 246 through an arc greater than 90 degrees and applying a lesser tension to the pull wire 252 will bend the deformable portion 246 through an arc of less than 90 degrees. However, it is noted that the application of excessive tension may shrink the volume of the second shape, e.g., pulling the deformable portion 246 into a tighter spiral. Those skilled in the art will understand that the user may achieve a desired amount of bending (i.e., to attain a desired shape of the deformable portion 246) by increasing the applied tension until the deformable portion 246 extends in a second shape occupying an area sufficient so that contact between the deformable portion 246 and/or the distal end 244 of the catheter 242 and the small bowel anchors the catheter 242 in a relatively fixed position within the small bowel.

Similar to the preceding embodiments, the catheter device 240 can be used as a feeding tube or to assist an EUS-GE procedure. To remove the catheter device 240 from the patient, the tension can be released from the pull wire to transition the catheter device 240 back into the first shape. The catheter device 240 may then be withdrawn proximally from the patient's body.

In another embodiment, a deformable catheter can comprise multiple Tome-style bending sections to create different (combined) second shapes, e.g., larger shapes for retention and/or a greater degree of bending. Sequential deployment of multiple bending sections could be achieved by altering the length of the bending sections. Longer sections would be more unstable and deploy first, and shorter sections would deploy later.

FIG. 8 shows a catheter device 260 comprising a pull wire 272 for transitioning a catheter 262 to an expanded state (second shape) according to a sixth example of these exemplary embodiments. The catheter 262 assumes an insertion state (first shape), e.g., a straight shape typical of a simple catheter, when no tension is applied to the pull wire 272. The catheter 262 extends from a proximal end (not shown) to a distal end 264 with an atraumatic distal tip and includes a distal portion comprising holes (not shown) for discharging a fluid passed through the lumen of the catheter, similar to the preceding embodiments.

In this example, the catheter 262 comprises a first deformable portion 266 adjacent to the distal end 264 and a second deformable portion 267 adjacent to the first deformable portion. Each of the deformable portions 266, 267 in this embodiment is configured to transition between a first shape and a second shape by the application and/or removal of tension to the pull wire 272. Each deformable portion 266, 267 includes a respective slit in its wall (in a plane that is not visible in FIG. 8) extending along its length, e.g., a first slit in the first deformable portion 266 and a second slit in the second deformable portion 267, separated by a short length of catheter that does not have a slit. The catheter 262 in the first shape is passed through the GI tract such that the deformable portions 266, 267 are at a desired location within the small bowel.

A distal end 268 of the first slit provides an anchor position for a distal end 274 of the pull wire 272. The pull wire 272 runs loosely within the lumen of the catheter 262 from the anchor point at its distal end 274 to a proximal end (not shown) that can be tensioned by an operating physician. A proximal end 269 of the first slit, a distal end 270 of the second slit, and a proximal end 271 of the second slit all provide surfaces that restrict the translation of the pull wire 272 in two dimensions while allowing longitudinal motion thereover.

As shown in FIG. 8, when tension is applied to the pull wire 272, the first and second deformable portions 266, 267 of the catheter 262 can transition into the second shape or expanded shape. Specifically, the first slit in the first deformable portion 266 and the second slit in the second deformable portion 267 allow the pull wire 272 to pass outside the lumen of the catheter 262 such that tensioning the pull wire 272 to apply a distal force at the anchor position at the distal end 268 of the first slit (adjacent to the distal end 264 of the catheter 262), bends the catheter 262 along the portions comprising the slits. The pull wire 272 is drawn proximally over the proximal end 269 of the first slit, within a short length of the catheter 262 to the distal end 270 of the second slit, over the distal end 270 of the second slit, outside the second deformable portion 267, and over the proximal end 271 of the second slit.

In this example, the second shape of each of the deformable portions 266, 267 comprises a substantially 90 degree bend, such that the total bending of the combined deformable portions 266, 267 is substantially 180 degrees. However, it should be understood that different bend radii can be applied based on different applications of tension by a user. Further, as described above, the slits of the deformable portions 266, 267 can be sized and shaped differently such that one of the deformable portions 266, 267 (e.g., the first deformable portion 266) bends more under the tension of the pull wire before (e.g., is more unstable than) the other one of the deformable portions 266, 267 (e.g., the second deformable portion 267). A desired amount of bending can be applied such that, in the second shape, the deformable portions 266, 267 occupy an area sufficient large so that contact between the first deformable portion 266 and/or the distal end 264 of the catheter 262 and the small bowel anchors the catheter 262 in a relatively fixed position within the small bowel.

Similar to the preceding embodiments, the catheter 262 can be used as a feeding tube or to assist an EUS-GE procedure. To remove the catheter 262 from the patient, the tension is released from the pull wire 272 to transition the catheter 262 back to the first shape.

In another aspect of these exemplary embodiments, a deformable catheter can incorporate surface features for anchoring to or otherwise engaging an inner surface of the small bowel (duodenum and/or jejunum) when the catheter expands or otherwise changes shape to contact the inner surface of the small bowel. The surface features can comprise barbs, micropatterns, or other features for adhering to the inner wall of the small bowel to increase retention and positioning of the catheter. The surface features, e.g., protrusions, may be of varying shapes and sizes according to various designs.

In some embodiments, small cuts or slits can be made in the catheter so that, when the catheter bends, portions of the catheter adjacent to (e.g., shaped by) the cuts protrude from the catheter. In these embodiments, the catheter bends or coils in a spiral shape, e.g., similar to the catheters 210, 230 described above with regard to FIGS. 4, 6b, such that one surface of the bending portion is stretched (e.g., the outer surface of the coil) while the opposing surface is compressed. The cuts are formed on the outer surface such that tensioning the surface causes the cuts to separate further. In combination with the bending of the surface, the sections adjacent to the cuts can protrude.

In one example, a series of transverse cuts can be made at locations along the length of the deformable portion, forming sections between the cuts. When the deformable portion is coiled a proximal end of each of the sections (proximal side of the cuts) protrudes radially away from the distal side of the cut. However, the degree of protrusion may be limited when only transverse cuts are used. In another example, the cuts can be curved, e.g., include longitudinal cuts on the sides of the transverse cuts, such that the shape of the sections is more clearly defined and a greater degree of protrusion can result. The protrusions may have relatively sharply defined corners and/or edges such that, when extended with the catheter in the second shape, barbs are formed for engaging the inner surfaces of the small bowel.

FIG. 9a shows a catheter 280 comprising slits 287 in the walls of the catheter 280 forming sections 288 in an insertion state (first shape) according to a seventh example of these exemplary embodiments; FIG. 9b shows the catheter 280 of FIG. 9a in an expanded state (second shape) wherein barbs 289 are formed by the sections 288 according to the seventh exemplary embodiment.

As shown in FIG. 9a, the catheter 280 initially assumes an insertion state (first shape), e.g., straight shape typical of a simple catheter. The catheter 280 extends from a proximal end (not shown) to a distal end 282 with an atraumatic distal tip and includes a distal portion comprising holes 284 for discharging a fluid passed through the lumen of the catheter 280 to a target site within the body. The catheter 280 comprises a deformable portion 286 configured to transition between a first shape and a second shape, as shown in FIG. 9b. It should be understood that the holes 284 can be provided at various locations similar to those described in any of the preceding embodiments. In this example, the holes 284 are located on the deformable portion 286. In this example, the second shape is a spiral coil. In view of the barb features to be described below, the expanded shape may comprise only a single full rotation or less. In the insertion state, the sections 288 formed by the slits 287 lie flat.

As shown in FIG. 9b, when the catheter 280 bends to assume the second shape, the slits 287 cause proximal ends of the sections 288 to extend radially away from the remainder of the catheter 280, forming barbs 289. The barbs 289 can engage the inner walls 131 of the small bowel 130.

It should be understood that transitioning the catheter 280 between the first shape and the second shape (spiral distal end) can be effectuated by any of the mechanisms described previously, e.g., by insertion/removal of a guidewire, by applying a voltage to an EAP strip (e.g., located on a side of the catheter 280 opposite the slits 287), or by pull wire in a Tome-style device.

In another embodiment, a catheter incorporates a micropatterned outer surface. Micropatterns such as micro-scale pillars have been studied extensively as effective tissue adhesives. Micropatterning the catheter or NJ tube allows the surface of the tube to be more adhesive through a combination of force (interlock) and suction, increasing its ability to adhere to the walls of the jejunum. The micropattern could take several forms depending on the shape of the catheter. If the NJ tube is capable of coiling such that it “expands” to exert some force on the jejunum wall (either by manufacture or through one of the exemplary embodiments), the micropattern could be designed to focus on adhesion through polymer-tissue interlock. If the catheter creates less force on the jejunum (if the tube maintained a straight rather than coiled configuration, for example), the micropattern could be designed to focus more on suction and taking advantage of the wet/mucous environment of the small intestine to generate adhesion.

FIG. 10a shows a catheter 290 comprising a first micropatterned surface 292 according to an eighth example of these exemplary embodiments. In this example, the catheter 290 comprises a helical coil shape, e.g., similar to the catheters 202, 220 of FIGS. 3c, 5b. The first micropatterned surface 292 comprises substantially rectangular protrusions.

FIG. 10b shows a second micropatterned surface 294 according to a ninth example of these exemplary embodiments. The second micropatterned surface 294 comprises protrusions that flair radially.

FIG. 10c shows a third micropatterned surface 296 according to a tenth example of these exemplary embodiments. The third micropatterned surface 296 comprises protrusions that are rounded, e.g., in an egg-like shape.

FIG. 10d shows a fourth micropatterned surface 298 according to an eleventh example of these exemplary embodiments. The fourth micropatterned surface 298 comprises protrusions with a mushroom top.

It should be understood that the micropatterned surface can be used in combination with a number of the preceding embodiments, including, e.g., the catheters 202, 210, 220, 230, 240, 260 or 280.

In another embodiment, the catheter can comprise barbs for anchoring to the distal side of the pylorus. The barbs can be strategically placed and oriented such that they will lay flat in order to allow ease of passage through narrow body lumens such as the esophagus and the pylorus. Once the barbs pass the stricture they can open and thus provide interference with the tissue (stricture) to mitigate migration of the catheter in the reverse direction (towards the stomach).

FIG. 11a shows a catheter 300 comprising a barbed portion 302 according to a twelfth example of these exemplary embodiments. The catheter 300 extends from a proximal end (not shown) to a distal end 306 with an atraumatic distal tip (as shown in FIG. 11b). The barbed portion 302 may include one or more anti-migration barbs 304. In this example, the barbed portion 302 comprises two barbs 304. However, one barb or greater than two barbs may also be used. The barbs 304 may be disposed on opposing sides of a same location along the length of the catheter 300.

The barbs 304 are oriented to project radially and proximally from the catheter 300 in their natural shape (e.g., with no forces being applied thereto). Thus, as the catheter 300 is advanced through the body, narrow lumens of the body will contact the barbs 304 first at their distal side (adjacent to the catheter 300), applying a radially inward force so that the barbs 304 lie flat. When the barbs 304 are outside the narrow lumen they can project back into their natural shape.

FIG. 11b shows the catheter 300 of FIG. 11a deployed in the anatomy 120 of FIG. 1b according to the twelfth exemplary embodiment. The barbs 304 are passed through the stricture 138 so that the ends of the barbs 304 engage the distal side of the pylorus 128. The location of the barbs 304 along the length of the catheter 300 can be selected such that, when the barbs 304 are anchored to the pylorus, the distal end 306 of the catheter is in a desired location in the small bowel. Similar to the preceding embodiments, a distal portion (not labeled) of the catheter 300 can comprise holes (not labeled) for discharging a fluid passed through the lumen of the catheter 300.

For removal, the catheter 300 may be pulled proximally with enough force that the barbs 304 are overcome and deformed, bending distally to the point that they lay flat against the tube and no longer provide restriction to proximal movement.

In another aspect of these exemplary embodiments, the catheter can expand with Malecot style features. This can be beneficial for both feed tube migration reduction and keeping said catheter in position for injecting contrast during gastrojejunostomy.

Catheters with Malecot anchors generally refer to catheters comprising longitudinal cuts forming arms/sections between adjacent cuts. For example, four longitudinal cuts of predetermined length may be spaced equally around the circumference of the catheter, thus forming four sections along this length. These sections remain straight during insertion and can then be deployed by pulling a distal portion of the catheter (e.g., distal to the Malecot sections) proximally (e.g., from a position proximal to Malecot sections) so that the Malecot sections are compressed and flair out, e.g., into “arms.” In one example, the Malecot sections can be deployed by pulling an internal catheter connected to the distal tip of an external catheter (the external catheter comprising the Malecot sections) proximally, as described in further detail below in FIGS. 13b-c. The Malecot sections expand to anchor the catheter in place.

FIG. 12 shows a catheter 310 comprising a Malecot-type anchor 312 according to one example. The Malecot-type anchor 312 of this example includes four arms 314 defined by four slits or cuts in the catheter 310. An internal catheter 316 can be pulled or pushed to expand or contract the arms 314. In the example shown in FIG. 12, the arms 314 are expanded.

According to various exemplary embodiments, Malecot-style cuts are configured for the exemplary catheters to achieve various aims with regard to flexibility, expansion and accessibility to the interior volume of the catheter. In one example, a greater number of arms provides greater flexibility such that a lesser force would be needed to expand the arms. In another example, longer sections can provide a larger volume when the arms expand.

In some exemplary embodiments, a single Malecot-type mechanism can be used. The Malecot arms can expand to contact the inner wall of the small bowel to create friction against the wall of the small bowel and increase the amount of force required to make the catheter move. Different from the way a typical Malecot is pulled into a short, fully-compressed anchor, catheters according to the exemplary embodiments could be designed to form more of an elongated balloon shape, providing enough drag against the bowel wall to prevent unintentional movement but also providing a fluid pathway for flooding the lumen.

FIG. 13a shows a catheter 320 comprising Malecot-type features in an insertion state (first shape) according to a thirteenth example of these exemplary embodiments; FIG. 13b shows a catheter 340 comprising the catheter 320 of FIG. 13a and further including a film cover 342 in an expanded state (second shape) according to a fourteenth example of these exemplary embodiments; FIG. 13c shows a catheter 350 comprising the catheter 320 of FIG. 13a and further including a partial film cover 352 in an expanded state (second shape) according to a fifteenth example of these exemplary embodiments. It should be understood that the catheter 320 of FIG. 13a does not employ the use of a film as described in FIGS. 13b-c, however, the expansion of the catheter 320 will be described with regard to these Figures.

As shown in FIG. 13a, the catheter 320 extends from a proximal end (not shown) to a distal end 322 with an atraumatic distal tip. Near the distal end 322 is a deformable portion 324 configured to transition between a first shape and a second shape. The deformable portion 324 may comprise Malecot-type features. In this example, the deformable portion 324 comprises a length of the catheter 320 in which longitudinal slits 326 have been made to form longitudinal sections 328 in the catheter 320.

In this example, the deformable portion 324 is adjacent to the distal end 322 with a short distal portion 330 between the deformable portion 324 and the distal tip, however, in other embodiments the deformable portion 324 can be located further proximally along the catheter 320. In its natural state (e.g., when no external forces are applied), the catheter 320 is in the first shape, e.g., an insertion state, in which the longitudinal sections 328 lie flat so that the catheter 202 can be passed through the GI tract. The catheter 320 can be passed through the GI tract such that the deformable portion 324 is at a desired location within the small bowel.

The catheter 320 can be transitioned into the second shape by an inner member 332 extending through the lumen of the catheter 320, as shown in FIGS. 13b-c. The inner member 332 can be, e.g., an inner catheter. The inner member 332 extends from a proximal end (not shown) to a distal end (not shown) anchored to the catheter 320 within the distal portion 330 and/or distal tip. The proximal end of the inner member 332 can be pulled so that the distal end 322 is pulled proximally relative to the proximal side of the deformable portion 324, which can be maintained in its current position.

When the inner member 332 is pulled, the deformable portion 324 of the catheter 320 transitions into the second shape (or expanded shape). In this example, the longitudinal sections 328 compress at their ends to bend radially outward, forming arms 334. The resulting shape is an elongated balloon-type shape and can be considered a radial expansion and/or increased volume, relative to the cross section of the catheter 320 in the first shape, such that the outer surface of the arms 334 can be brought into contact with the inner walls of the small bowel. In the second shape, the contact between the deformable portion 324 and the small bowel causes the catheter 320 to remain in relatively fixed position within the small bowel.

The deformable portion 324 can contact the inner wall of the small bowel and fluids passed through the lumen of the catheter 320 (outside the inner member 332) can be discharged, e.g., by the spaces between the arms 334. It is noted that, in the example of FIG. 13a, the catheter 320 may be used as a feeding tube and the deployment location of the deformable portion 324 may be selected accordingly to provide nutrients that will be digested at a proper location, e.g., at the beginning of the jejunum. In other embodiments, the catheter 320 can include holes for discharging the fluid, e.g., proximal to the deformable portion 324, and the deployment location of the deformable portion 324 may be adjusted accordingly. In these applications, the catheter 320 can remain in the expanded shape for retention in the small bowel over an extended period of time (weeks, months, etc.). However, it is noted that the sizes and number of parts involved in this embodiment may decrease flow through the catheter such that feeding tube applications may not be feasible.

In a preferred embodiment, the catheter may be deployed so that the deformable portion 324 is placed further distally, e.g., within the jejunum, so that fluid can be applied at a further distal location, e.g., to assist an EUS-GE procedure. If the catheter 320 is used for EUS-GE purposes, contrast and/or other fluid (e.g., saline) can be applied via the spaces between the arms 334 to flood the small bowel, which would migrate distally over time. Additionally, the expanded Malecot could expand the small bowel and create space for a needle entering through the sidewall. Thus, the needle can penetrate the jejunum and the expanded Malecot will maintain its expanded balloon-shaped target that is non-puncturable, which provides an advantage over devices employing only a balloon, which would be punctured by the needle and then deflate such that the jejunum would no longer be expanded. It is further noted that, in some embodiments, the inner member 332 or the expanded arms 334 can comprise echogenic features such that these features are visible without contrast injection. In this case, fluid such as saline can be discharged to enhance visibility of the inner member 332 and/or arms 334.

The catheter 320 can be transitioned back into the insertion shape by pushing the inner member 332 to disengage the deformable portion 324 from the small bowel to remove the catheter 320 from the patient anatomy.

As described above, the catheter 320 can provide a fluid pathway for contrast or saline to flood the lumen. The design of the catheter 320 can be enhanced for EUS-GE applications by adding a film cover for retaining saline/contrast in the volume defined by the Malecot sections.

In one method, a target is created for the gastro-jejunostomy procedure by using a film covering the struts completely, similar to a balloon, where the expanded film in the small bowel can retain contrast/saline for targeting by a needle through the stomach. The balloon-like target provides assurance that the correct bowel loop is targeted as well as expands the collapsed bowel lumen. Although the film would be punctured by the needle, thus allowing the release of the fluid, the expanded bowel loop can still be retargeted after puncture because the catheter is continuing to expand the lumen and some fluid will be retained. A needle puncture through the expanded lumen does not cause the device to collapse as the needle will go through the space between the arms and into the created space.

As shown in FIG. 13b, the catheter 340 comprises the catheter 320 and further includes a film cover 342 that can expand when the arms 334 expand. The film cover 342 may be filled with saline/contrast to provide a target for EUS-GE procedures, as described above.

In another embodiment, a partial distal film cover covering part of the second shape, e.g., 40-70% of the distal end of the expanded arms 334, can be used. This partial film cover is configured to capture some of the saline/contrast being added to the lumen. The force of the arms against the lumen wall would create a seal and mostly prevent the fluid from being lost to the distal bowel. This would slow the movement of the liquid compared to the bowel flooding, with no obstruction, currently in use.

As shown in FIG. 13c, the catheter 350 comprises the catheter 320 and further includes a partial film 352 on the distal part of the Malecot section that can expand as the arms 334 expand. The partial film 352 may capture some saline/contrast to retain the fluid at the target location. It is noted that a balloon-like film may also be used with the catheter 350, such that the full film can completely retain the fluid prior to puncture and, after it is punctured, the partial film 352 can retain some of the fluid that would otherwise be lost.

In certain situation it may be advantageous to grab the guidewire from the small bowel. After the needle has placed the guidewire and been withdrawn, pushing the structure back into its initial, closed position would capture a guidewire if it were placed within the expanded configuration.

In some exemplary embodiments, multiple Malecots can be formed in the catheter. While a single Malecot anchor can provide resistance to migration, multiple Malecots would provide greater resistance. In addition, the use of multiple Malecots offers additional functionality. Specifically, one or more Malecots could be deployed on either side of a stricture to resist migration in both proximal and distal directions. One issue with using multiple sections is control of deployment. However, each Malecots may be designed such that they may be deployed sequentially, and not all at once. This may be achieved by altering the number of arms for each Malecot portion. For example, a Malecot with more arms will deploy first. Since the arms are thinner and flatter, they provide less resistance individually and overall. For example, an NJ tube could be constructed with 2 sections, one with 4 arms and one with 6 arms. The 6-arm section could be deployed first, followed by the 4-arm section. Another way to achieve sequential deployment would be to alter the deployed size of the section. Larger sections with longer arms are less stable and will deploy at lower force than smaller sections with shorter arms.

FIG. 14a shows a catheter 360 comprising multiple Malecot-type features according to a sixteenth example of these exemplary embodiments. In this example, a first deformable portion 362 includes a greater number of arms than a second deformable portion 364. Accordingly, the first deformable portion 362 will expand under a compressive force lesser than the second deformable portion 364, thus deploying the anchors sequentially.

FIG. 14b shows a catheter 370 comprising multiple Malecot-type features according to a seventeenth example of these exemplary embodiments. In this example, a first deformable portion 372 includes longer arms than a second deformable portion 374. Accordingly, the first deformable portion 372 will expand under a compressive force lesser than the second deformable portion 374, thus deploying the anchors sequentially.

In some exemplary embodiments, spiral-cut Malecots can be formed in the catheter. Traditional Malecot catheters are designed so that the arms move and deflect in planes radiating from the longitudinal axis. This requires a longitudinal input action. By altering the geometry of the arms, the input mechanism may be similarly altered. If the geometry of the arms are spirals or helices, deployment may be achieved by either purely longitudinal input, purely rotational input (shown below), or a combination of the two. This provides more flexibility in the design of the proximal user controls.

FIG. 15 shows a catheter 380 comprising spiral cut Malecot-type features according to an eighteenth example of these exemplary embodiments. In this example, a deformable portion 382 comprises cuts that extend in helically spiral shapes such that the deformable portion 382 will expand under a rotational force.

In some exemplary embodiments, an auxetic foam may be used with the Malecot-type catheters of the preceding embodiments to achieve a further Malecot effect. This is particularly useful because the typical Malecot design depends on foreshortening of the catheter to achieve a change in the OD. With an auxetic material, an extending motion can also be utilized. Auxetic foam may be included in the core of the Malecot such that it helps expand when the material is activated. When an auxetic material is incorporated into the catheter, it functions like a balloon in obstructing the duct and holding the catheter in place, but acts like a Malecot in translation of force that creates expansion in a perpendicular direction.

FIG. 16a shows a conventional material 390 according to one example. The conventional material 390 in the shape of a cylinder compresses radially as it elongates under a pulling force.

FIG. 16b shows an auxetic material. 392 according to one example. The auxetic material 392 in the shape of a cylinder expands radially as it elongates under a pulling force.

In still another embodiment, a catheter may be capable of having the distal tip scrunched to create peaks for engaging the inner wall of the small bowel and valleys for folds of tissue to land in, enabling retention of the NJ catheter. In a first state, the catheter would be in a straight configuration (first shape), enabling it to be delivered along a guidewire to the target tissue site past the stricture. Once at the target tissue site the catheter would be forced into an expanded configuration (second shape). This could be done in various ways, e.g., by removing the guide wire, by a second drive wire in its own lumen, or by a twisting motion, similar to the various expansion mechanisms described in the preceding embodiments.

FIG. 17a shows a catheter 400 comprising a deformable portion 404 in an insertion state (first shape) according to a nineteenth example of these exemplary embodiments. The deformable portion 404 is adjacent to a distal end 402 of the catheter.

FIG. 17b shows the catheter 400 of FIG. 17a in an expanded state (second shape) according to the nineteenth exemplary embodiment. The second shape comprises peaks 406 and valleys 408. In the expanded configuration, the distal tip of the catheter would have a series of scrunched sections, such that multiple peaks 406 and valleys 408 are generated. These features not only make it difficult to migrate through the stricture but also provide features for folds in the jejunal tissue to track into. These peaks 406, however, would not impose significant enough traction that could cause complications that could occur during placement of a stent.

It may be noted by those knowledgeable in the art that any of the above embodiments may be combined in any manner not inconsistent with their operation and design to provide a system to enable ostomy management utilizing any or all of the characteristics of the various embodiments.

1-15. (canceled)

16. A device for administering a fluid to a patient anatomy, comprising:
a flexible catheter including a lumen extending therethrough, the catheter being sized and shaped to extend from a proximal end outside a human body to a distal end that can be guided to a target location within a gastrointestinal (GI) system, a distal portion of the catheter including one or more exit ports communicating with a first lumen for administering a fluid; and
a deformable portion of the catheter being formed so that, in a first state, the deformable portion assumes a first shape suitable for guiding the catheter to the target location and, in a second state, the deformable portion assumes a second shape in which the deformable portion curves to extend in at least one radial direction for engaging an inner wall of the target location to anchor the catheter in the target location while administering the fluid via the lumen.

17. The device of claim 16, further comprising:
a guidewire sized and shaped to extend through the lumen of the catheter so that, when the guidewire is inserted within the deformable portion, the deformable portion assumes the first shape and, when the guidewire is removed, the deformable portion assumes the second shape.

18. The device of claim 17, wherein the guidewire comprises a rigidity greater than that of the deformable portion such that the deformable portion conforms to a shape of the guidewire when the guidewire is within the lumen.

19. The device of claim 18, wherein the second shape of the deformable portion comprises a helical coil or a spiral coil, the second shape being a natural shape of the deformable portion,
wherein inserting the guidewire within the deformable portion straightens the deformable portion into the first shape.

20. The device of claim 16, further comprising:
an electroactive polymer (EAP) incorporated into the catheter,
wherein the catheter transitions between the first and second states when a voltage is applied to the EAP or when the voltage is removed from the EAP.

21. The device of claim 16, further comprising:
a pull wire extending from a proximal end outside the human body through the lumen of the catheter to a distal end anchored to the catheter at or near the distal end of the catheter,
wherein the catheter includes a slit extending along the deformable portion such that, when tension is applied to the pull wire, the deformable portion curves as a distal portion of the pull wire translates through the slit to an exterior of the catheter to transition the deformable portion into the second state.

22. The device of claim 16, wherein the deformable portion comprises a series of slits extending at least transversely across the catheter, each slit forming a section of the catheter distal to the slit and wherein the second shape comprises a spiral coil such that, when the deformable portion transitions into the second shape, proximal ends of the sections protrude to form barbs for engaging the inner wall of the target location.

23. The device of claim 16, wherein the deformable portion comprises a micropatterned surface for adhering to the inner wall of the target location.

24. The device of claim 16, wherein the deformable portion comprises multiple longitudinal slits forming multiple longitudinal sections, the device further comprising:
an inner member extending through the lumen of the catheter, a distal end of the inner member fixed to the catheter distal to the deformable portion,
wherein pulling the inner member forces the distal end of the catheter proximally such that the second shape comprises a radial expansion of the sections into arms for engaging the inner wall of the target location.

25. The device of claim 24, further comprising:
a film covering the deformable portion in the second shape to fully retain the fluid discharged via the deformable portion or a partial film covering a distal part of the deformable portion to partially retain the fluid.

26. The device of claim 24, wherein the catheter comprises a further deformable portion comprising multiple longitudinal slits forming multiple longitudinal sections, wherein the deformable portion comprises a greater number of slits and sections than the further deformable portion,
wherein, when the inner member is pulled, the deformable portion and the further deformable portion deploy in sequence.

27. The device of claim 24, wherein the catheter comprises a further deformable portion comprising multiple longitudinal slits forming multiple longitudinal sections, wherein the deformable portion is longer than the further deformable portion,
wherein, when the inner member is pulled, the deformable portion and the further deformable portion deploy in sequence.

28. The device of claim 16, wherein the deformable portion comprises multiple slits in a helical spiral pattern forming multiple sections, the device further comprising:
an inner member extending through the lumen of the catheter, a distal end of the inner member fixed to the catheter distal to the deformable portion,
wherein rotating the inner member forces the distal end of the catheter to rotate such that the second shape comprises a radial expansion of the sections into arms for engaging the inner wall of the target location.

29. The device of claim 16, further comprising:
an auxetic material within the deformable portion that expands to expand the deformable portion into the second shape by an elongation or extension applied to the auxetic material.

30. The device of claim 16, wherein the second shape comprises a scrunched shape creating peaks for engaging the inner wall of a small bowel and valleys for receiving folds of tissue to retain the catheter in position.

31. A method for administering a fluid to a patient anatomy, comprising:
guiding a flexible catheter to a target location within a gastrointestinal (GI) system, the catheter including a lumen extending therethrough, the catheter being sized and shaped to extend from a proximal end outside a human body to a distal end, a distal portion of the catheter including one or more exit ports communicating with a first lumen for administering a fluid, a deformable portion of the catheter being formed so that, in a first state, the deformable portion assumes a first shape suitable for guiding the catheter to the target location; and
transitioning the catheter into a second state wherein the deformable portion assumes a second shape in which the deformable portion curves to extend in at least one radial direction for engaging an inner wall of the target location to anchor the catheter in the target location while administering the fluid via the lumen.

32. The method of claim 31, wherein the deformable portion assumes the first shape when a guidewire is extended through the lumen of the catheter and, when the guidewire is removed, the deformable portion assumes the second shape, the guidewire comprising a rigidity greater than that of the deformable portion such that the deformable portion conforms to a shape of the guidewire when the guidewire is within the lumen; or
wherein an electroactive polymer (EAP) is incorporated into the catheter, wherein the catheter transitions between the first and second states when a voltage is applied to the EAP or when the voltage is removed from the EAP; or
wherein a pull wire extends from a proximal end outside the human body through the lumen of the catheter to a distal end anchored to the catheter at or near the distal end of the catheter, wherein the catheter includes a slit extending along the deformable portion such that, when tension is applied to the pull wire, the deformable portion curves as a distal portion of the pull wire translates through the slit to an exterior of the catheter to transition the deformable portion into the second state.

33. The method of claim 31, wherein the deformable portion comprises a series of slits extending at least transversely across the catheter, each slit forming a section of the catheter distal to the slit and wherein the second shape comprises a spiral coil such that, when the deformable portion transitions into the second shape, proximal ends of the sections protrude to form barbs for engaging the inner wall of the target location; or
wherein the deformable portion comprises a micropatterned surface for adhering to the inner wall of the target location.

34. The method of claim 31, wherein the deformable portion comprises multiple longitudinal slits forming multiple longitudinal sections, an inner member extends through the lumen of the catheter, a distal end of the inner member fixed to the catheter distal to the deformable portion,
wherein pulling the inner member forces the distal end of the catheter proximally such that the second shape comprises a radial expansion of the sections into arms for engaging the inner wall of the target location.

35. The method of claim 34, wherein a film covers the deformable portion in the second shape to fully retain the fluid discharged via the deformable portion or a partial film covers a distal part of the deformable portion to partially retain the fluid.
